(19) United States
(12) Patent Application Publication
(10) Pub. No.: US 2021/0290316 A1
(43) Pub. Date: Sep. 23, 2021

(54) System And Method For Determining A Trajectory Of An Elongated Tool

(71) Applicant: NDR Medical Technology Pte. Ltd., Singapore (SG)

(72) Inventors: Ka Wei NG, Singapore (SG); Jin Quan GOH, Singapore (SG)

(21) Appl. No.: 17/262,520

(22) PCT Filed: Jul. 24, 2018
(86) PCT No.: PCT/SG2018/050365

§ 371 Date: Jan. 22, 2021

(51) Int. Cl. A61B 34/20; A61B 90/00

(52) U.S. Cl. CPC A61B 34/20; A61B 90/37; A61B 2034/107; A61B 2090/3762; A61B 2090/374

(57) ABSTRACT

Systems and methods for determining a trajectory of an elongated tool and for striking the target using the elongated tool are disclosed. The system for determining a trajectory of an elongated tool includes a memory module configured to receive imaging data of a preliminary 3-dimensional (3D) image of a body from a 3D imaging device, the body comprising an opaque body surface, a target and at least one occlusion between the body surface and the target, and a processor communicatively coupled with the memory module. The processor is configured to process the preliminary 3D image of the body from the 3D imaging device to obtain location data of the target, body surface and at least one occlusion; and based on a location of the target relative to the body surface and at least one occlusion, determine at least one trajectory for the elongated tool to strike the target.

### FIELD OF INVENTION

The present invention relates broadly to a system and a method for determining a trajectory of an elongated tool.

### BACKGROUND

There are several ways for insertion of a needle into a patient's body in a surgery. For example, the surgeon can perform the surgery manually by placing one end of the needle on a patient's skin, and based on real-time imaging data repeatedly tilting the other end of the needle to establish an alignment between the needle and the target. Using this method, the patient and surgical crew may be exposed to an excessive amount of radiation which could pose potential health hazards.

Medical instruments such as a robotic arm and a flexible needle have been introduced to automate the surgical procedure. However, most of these instruments merely mimic the manual process with the remote control of a clinician. Thus, the surgical procedures performed using these instruments are still prone to human errors that may compromise the outcome of the surgery.

Human errors in a surgical procedure may induce complications such as internal haemorrhage and pneumothorax. Due to these human errors, the needle may have to be withdrawn for the entire procedure to be repeated. This may aggravate the condition of a patient as multiple punctures of the patient's body may increase the risks to the patient.

A need therefore exists to provide a system and a method for striking an occluded target that seek to address at least one of the problems above or to provide a useful alternative.

### SUMMARY

According to a first aspect of the present invention, there is provided a system for determining a trajectory of an elongated tool, the system comprising:

a memory module configured to receive imaging data of a preliminary 3-dimensional (3D) image of a body from a 3D imaging device, the body comprising an opaque body surface, a target and at least one occlusion between the body surface and the target;

a processor communicatively coupled with the memory module, wherein the processor is configured to:
process the preliminary 3D image of the body from the 3D imaging device to obtain location data of the target, body surface and at least one occlusion; and
based on a location of the target relative to the body surface and at least one occlusion, determine at least one trajectory for the elongated tool to strike the target.

The processor may further be configured to determine at least one tool insertion point on the body surface to determine the at least one trajectory for the elongated tool to strike the target.

The processor may further be configured to determine the insertion point on the body surface having the shortest distance between the body surface and the target.

The processor may further be configured to determine the insertion point on the body surface such that a line between the insertion point and the target bypasses the at least one occlusion.

The processor may further be configured to determine coordinates of a centroid of the target to obtain location data of the target.

The system may further comprise a display device coupled to the processor, wherein the processor may further be configured to simulate a trajectory of the elongated tool based on the determined at least one trajectory for display on the display device.

According to a second aspect of the present invention, there is provided a system for striking a target using an elongated tool, the system comprising;

a 3D imaging device;

the system as defined in the first aspect connected to the 3D imaging device;

an adjustment mechanism configured to adjust an angular orientation of the elongated tool relative to the insertion point; and

an actuator coupled to the adjustment mechanism for moving the adjustment mechanism according to signals received from the processor,

wherein the 3D imaging device is further configured to capture a real-time 3D image of the body and the elongated tool,

wherein the processor is further configured to control the adjustment mechanism to align a longitudinal axis of the elongated tool with a selected trajectory based on the real-time 3D image, the processor further configured to calculate a striking distance between the insertion point and the target based on location data of the insertion point and the target; and

wherein the actuator is configured to drive the elongated tool toward the target based on the angular orientation of the elongated tool at alignment and the calculated striking distance.

The processor may further be configured to associate the real-time 3D image with the preliminary 3D image, to align the elongated tool to the selected trajectory.

The 3D imaging device may comprise at least one selected from a group consisting of a magnetic resonance imaging (MRI) machine, a computerized tomography (CT) scanner and a fluoroscope.

The adjustment mechanism may comprise a base and a platform, wherein the platform is configured to be parallel to the base.

The adjustment mechanism may further comprise a plurality of arms linking the base with the platform, the plurality of arms being configured to move the platform along a plane parallel to the base to adjust the angular orientation of the elongated tool relative to the insertion point.

The platform may comprise a ball joint compliance for supporting the elongated tool, the ball joint compliance comprising a hole configured to allow sliding movement of the elongated tool therethrough.

The adjustment mechanism may further comprise a tool holder detachable from the platform.

According to a third aspect of the present invention, there is provided a method for determining a trajectory of an elongated tool, the method comprising the steps of:

receiving a preliminary 3-dimensional (3D) image of a body, the body comprising an opaque body surface, a target and at least one occlusion between the body surface and the target;

processing the preliminary 3D image of the body from the 3D imaging device to obtain location data of the target, body surface and at least one occlusion; and

based on a location of the target relative to the body surface and at least one occlusion, determining at least one trajectory for the elongated tool to strike the target.

The step of determining the at least one trajectory for the elongated tool to strike the target may comprise determining at least one tool insertion point on the body surface.

The step of determining the at least one tool insertion point on the body surface may comprise determining the insertion point having the shortest distance between the body surface and the target.

The step of determining the at least one tool insertion point on the body surface may comprise determining the insertion point on the body surface such that a line between the insertion point and the target bypasses the at least one occlusion.

The step of processing the preliminary 3D image of the body to obtain location data of the target may comprise determining coordinates of a centroid of the target.

The method may further comprise the step of simulating a trajectory of the elongated tool based on the determined at least one trajectory for display on a display device.

According to a fourth aspect of the present invention, there is provided a method of striking a target using an elongated tool, the method comprising the steps of:

determining at least one trajectory for the elongated tool to strike the target using the method as defined in the third aspect;

receiving a real-time 3D image of the body and the elongated tool;

aligning a longitudinal axis of the elongated tool with a selected trajectory based on the real-time 3D image;

calculating a striking distance between the insertion point and the target based on location data of the insertion point and the target; and

advancing the elongated tool toward the target according to the calculated distance.

The step of aligning the longitudinal axis of the elongated tool with the selected trajectory may comprise associating the real-time 3D image with the preliminary 3D image.

### BRIEF DESCRIPTION OF THE DRAWINGS

Embodiments of the invention are provided by way of example only, and will be better understood and readily apparent to one of ordinary skill in the art from the following written description and the drawings, in which:

FIG. 1A shows a schematic diagram illustrating a set-up for determining a trajectory of an elongated tool according to an example embodiment.

FIG. 1B shows the connections between components of the set-up of FIG. 1A.

FIG. 2A shows a perspective view of an adjustment mechanism suitable for use in the system of FIGS. 1A and 1B.

FIG. 2B shows a front view the adjustment mechanism of FIG. 2A.

FIG. 2C shows two perspective views illustrating the use of a tool holder of the adjustment mechanism of FIG. 2A.

FIG. 2D shows an adjustment of the surgical tool using the adjustment mechanism of FIG. 2A.

FIG. 2E shows another example configuration of the adjustment mechanism of FIG. 2A during operation.

FIG. 3 shows a flowchart illustrating a treatment process of a lesion using the set-up of FIGS. 1A and 1B.

FIG. 4 shows a transverse plane view of lungs on a CT scan.

FIG. 5 shows the segmentation process of a CT scan using the system of FIGS. 1A and 1B.

FIG. 6 shows the centroid location of the lesion in a segmented view of FIG. 5.

FIG. 7A shows first illustration of a lesion in 3D voxel grids according to an example embodiment.

FIG. 7B shows second illustration of a lesion in 3D voxel grids according to an example embodiment.

FIG. 8 shows the determination of trajectories of the surgical tool to strike a lesion according to an example embodiment.

FIG. 9 shows a schematic diagram illustrating a computer suitable for implementing the system and method of the example embodiments.

### DETAILED DESCRIPTION

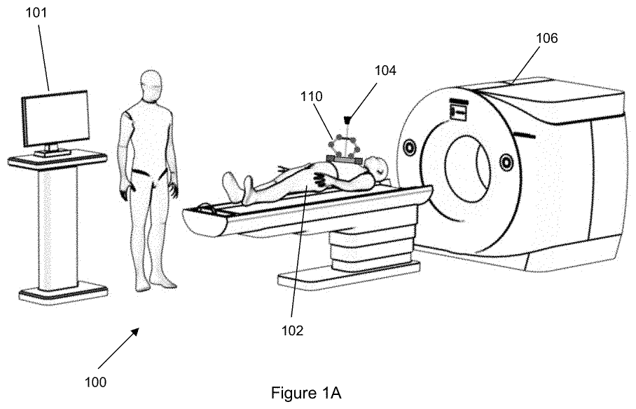

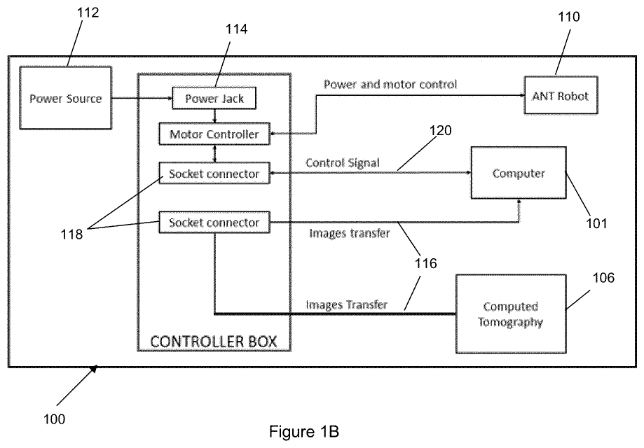

FIG. 1A shows a schematic diagram illustrating a set-up 100 for determining a trajectory of an elongated tool according to an example embodiment. In the description that follows, the set-up 100 is used to perform a surgical operation on a patient's body 102. Further, the elongated tool used in the operation is represented as a surgical tool 104, such as a biopsy or ablation needle, for treatment of a lesion within an organ inside the body 102. It will be appreciated that the set-up 100 can also be used in applications other than biopsy and ablation treatments and with different body organs, such as kidney stone removal and vertebroplasty.

FIG. 1A shows a 3-dimensional (3D) imaging device 106 configured to capture a preliminary 3D image of the body 102. Some examples of the 3D imaging device 106 include magnetic resonance imaging (MRI) machine, computerized tomography (CT) scan and fluoroscope.

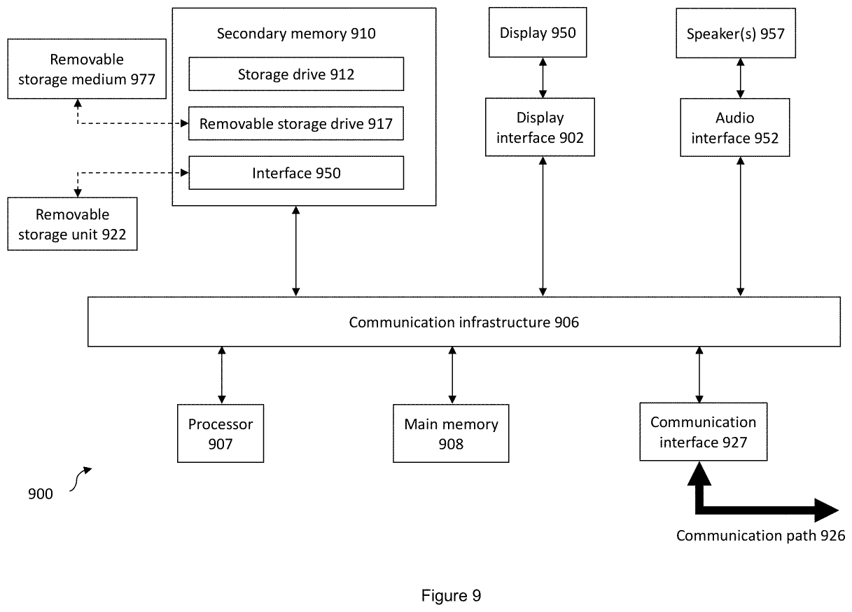

The set-up 100 includes a system 101 having a memory module (908 in FIG. 9, not shown in FIG. 1A) for receiving imaging data of the preliminary 3D image from the 3D imaging device. The system 101 further includes a processor (907 in FIG. 9, not shown in FIG. 1A) communicatively coupled with the memory module. The processor includes artificial intelligence (AI) software to process the preliminary 3D image from the 3D imaging device 106 to obtain location data of the lesion, body surface and at least one occlusion, e.g. other organs, bones, arteries inside the body 102. In oncologic imaging, a lesion typically has a richer blood supply than normal body cells which causes an identifiable shade to be generated on a 3D image, allowing the AI software to identify the image of the lesion. It will be appreciated that, instead of using AI, the lesion on the preliminary 3D image may also be manually selected by a clinician on a display device.

For example, the processor can automatically segment the preliminary 3D image to generate one or more segmented views to identify the lesion image on the segmented views. Next, the processor extracts location data of the lesion based on the lesion image. In an embodiment, the processor calculates centroid coordinates of the lesion in 3D voxel grids based on the generated segmented views. The processor is also configured to determine one or more sets of coordinates around the centroid coordinates in the 3D voxel grids for sample collections in a biopsy treatment or an ablation of the lesion.

Based on the centroid coordinates relative to the body surface and occlusions inside the body 102, the processor determines at least one trajectory for the surgical tool 104 to strike the lesion. In an embodiment, the processor determines at least one tool insertion point on the body surface for the insertion of the surgical tool 104. The insertion point of the surgical tool 104 is typically marked with an “X” mark on the skin of the patient's body 102. A tip of the surgical tool 104 can be placed on the mark when the angular orientation of the surgical tool 104 is being adjusted relative to the mark which acts as the pivot point.

The tool insertion point can be determined based on the distance between the body surface and the lesion. In an embodiment, the processor determines the insertion point on the body surface having the shortest distance between the body surface and the lesion. The processor is also configured to determine the insertion point on the body surface such that a line between the insertion point and the lesion bypasses the at least one occlusion. For example, the trajectory of the surgical tool 104 bypasses vital organs such as trachea, oesophagus and great vessels to avoid injuring the organs during the insertion of the surgical tool 104. Further, the trajectory also bypasses hard structures such as bones that can bend the biopsy needle.

The system 101 further includes a display device (not shown) coupled to the processor. The processor is further configured to simulate a trajectory of the surgical tool 104 toward the lesion based on the determined trajectory on the display device. By examining the simulation, the clinician is able to visualize the trajectory of the surgical tool 104 determined by the processor. If there is more than one trajectory determined by the processor, the clinician can be guided by the simulation in selecting a suitable trajectory for the surgery.

The system 101 further includes an adjustment mechanism, represented as robot 110, for adjusting an angular orientation of the surgical tool 104 relative to the insertion point. The robot 110 includes an actuator (not shown) for movement based on signals received from the processor. After the determination of the trajectory, the robot 110 together with the surgical tool 104 is mounted on the patient's body 102 at a desired place using an adhesive tape or gel.

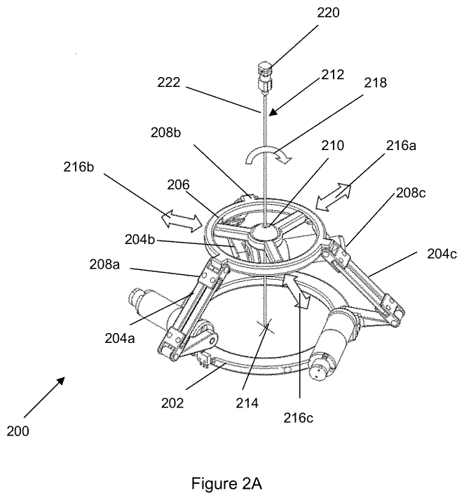

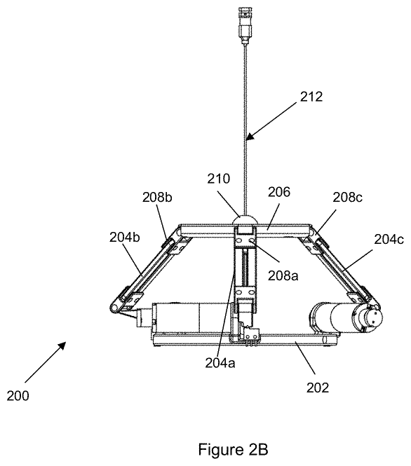

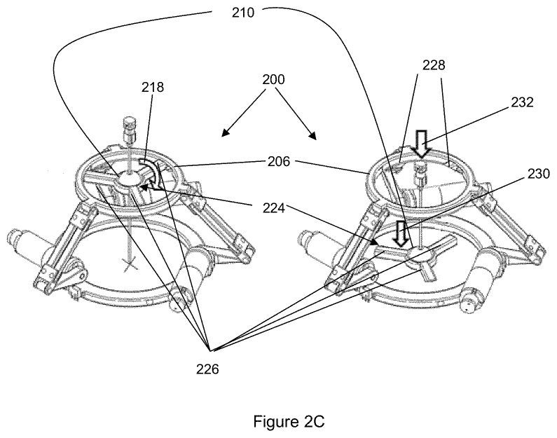

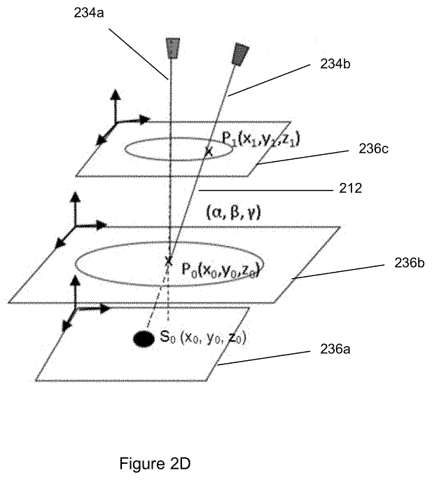

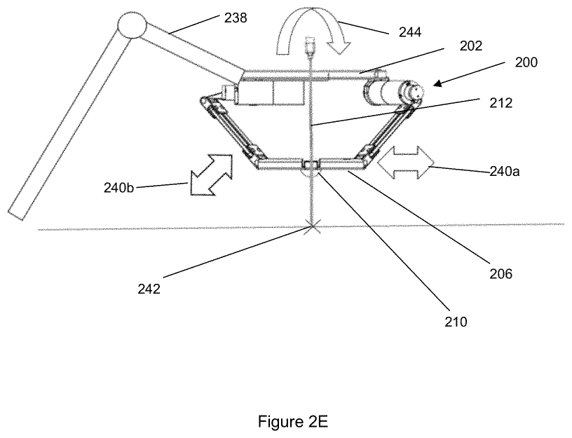

Advantageously, the robot 110 moves in tandem with the breathing movement of the body 102, minimizing skin and organ rupture during the operation. It will be appreciated that, instead of on the patient's body 102, a base of the robot 110 may be mounted, in an upward or inverted configuration, to a rigid structure above the patient's body 102 during a surgery such that the base is stationary. The configurations of the robot 110 during a surgery are explained in further detail below with respect to FIGS. 2A to 2E.

In one implementation, the 3D imaging device 106 then captures a real-time 3D image of the body 102 and the surgical tool 104. The processor receives the image data from the 3D imaging device 106 and fuses the real-time 3D image with the preliminary 3D image, followed by a calibration of the robot 110 to enhance the accuracy of the processor in controlling the robot 110 based on the real-time 3D image. The processor further controls the robot 110 to adjust the angular orientation of the surgical tool 104 relative to the insertion point, to align a longitudinal axis of the surgical tool 104 with the selected trajectory.

Next, the processor extracts location data of the insertion point of the surgical tool 104 from the real-time 3D image. Based on the location data of the pivot point and the lesion, a striking distance between the insertion point and the lesion is calculated. In an embodiment, the processor simulates a trajectory of the surgical tool 104 toward the lesion based on the calculated distance. If the simulation result is satisfactory, the clinician confirms to proceed with the insertion of the surgical tool 104 towards the lesion, either by automatic insertion controlled by the processor or manual insertion controlled by the clinician. The processor sends signals to the actuator to drive the surgical tool 104 toward the lesion based on the angular orientation of the surgical tool 104 at alignment and the calculated striking distance.

FIG. 1B shows the connections between components of the set-up 100 of FIG. 1A. As can be seen in FIG. 1B, the set-up 100 is a closed-loop control set-up which continues to operate until the process of striking the lesion is completed. The set-up includes a power source 112 which supplies power to other components of the set-up 100 via a power jack 114.

The system 101 (represented as a computer) is communicatively coupled with the 3D imaging device 106 (represented as a computed tomography system) using a wired connection such as an ethernet cable 116 joined using one or more socket connectors 118 that allows Digital Imaging and Communications in Medicine (DICOM). The system 101 is further connected to a motor controller via a serial cable 120 to transmit signals to the motor controller for adjustment and insertion of the surgical tool 104 using the robot 110 (represented as automatic needle targeting robot or ANT robot).

It will be appreciated that, instead of wired connections, the components in the set-up 100 can also be connected via wireless connections.

FIGS. 2A and 2B show perspective view and front view respectively of an adjustment mechanism 200 suitable for use in the system 101 of FIGS. 1A and 1B. The adjustment mechanism 200 comprises a base 202, in the form of an annular ring, and a plurality of arms, represented as first arm 204a, second arm 204b and third arm 204c. The arms 204a, 204b, 204c are connected to the base 202 at a substantially uniform angular distance from each other.

The adjustment mechanism 200 further comprises a raised platform 206 that is connected to end effectors 208a, 208b, 208c of the arms 204a, 204b, 204c respectively. The platform 206 is in the form of an annular ring and comprises a ball joint compliance 210 at the centre of the platform 206. The ball joint compliance 210 comprises a hole which holds a surgical tool 212 and allows sliding movement of the surgical tool 212. The ball joint compliance 210 further comprises a drive mechanism, in the form of a plunger (not shown), for holding and inserting the surgical tool 212 into a patient's body.

During operation, the base 202 is adhered to the patient's body. The arms 204a, 204b, 204c are actuated by at least one actuator (not shown) to coordinate with each other to adjust the position of the platform 206 and thus the orientation of the surgical tool 212 relative to the pivot point 214. The platform 206 typically moves at the same plane at a predetermined constant height relative to the base 202 during each operation, and the movement of the platform 206 relative to the base 202 is shown in FIG. 2A by arrows 216a, 216b, 216c. The height is normally determined at a calibration stage prior to the operation based on factors such as needle gauge, patient's physiology etc.

When the position of the platform 206 is adjusted by the arms 204a, 204b, 204c, the surgical tool 212 is held loosely by the plunger and the ball joint compliance 210, allowing the surgical tool 212 to pivot or swivel freely about the pivot point 214. This configuration allows tilting of the surgical tool 212 when the platform 206 is moved at the same plane, and the tilting of the surgical tool 212 is shown by arrow 218 in FIG. 2A.

The surgical tool 212 in the example embodiments comprises an adjustable stopper 220 mounted adjacent to an end 222 of the surgical tool 212 opposite the pivot point 214. Upon confirmation of the orientation of the surgical tool 212 and the depth of insertion, the position of the ball joint compliance 210 is locked and the stopper 220 is affixed to the surgical tool 212 with the distance between the stopper 220 and the ball joint compliance 210 being approximately equal to the insertion depth such that the depth of the insertion of the surgical tool 212 is restricted by the distance between the ball joint compliance 210 and the stopper 220. This configuration may advantageously restrict excessive insertion of the surgical tool 212 into the patient's body. Next, the plunger is actuated by the actuator to hold and insert the surgical tool 212 into the patient's body.

The structure of the adjustment mechanism 200 is typically made of light and rigid material. In an embodiment, the adjustment mechanism 200 is made of radiolucent material such that the 3D images provided by the 3D imaging device does not capture an image of the adjustment mechanism 200. In another embodiment, the parts of the adjustment mechanism 200 can be made of materials with different radiolucency. For example, the platform 206 of the adjustment mechanism 200 is made of radiopaque material, e.g. stainless steel, while other parts of the adjustment mechanism 200 are made of radiolucent material. In this instance, the image of the platform 206 is captured on the 3D image by the 3D imaging device and the location data of the platform 206 can be extracted from the 3D image for easy determination of the coordinates of the ball joint compliance and thus, the angular orientation of the surgical tool 212.

As the adjustment mechanism 200 has a simple structure and is relatively small in size, it can respond quickly to signals from the processor. Also, the configuration of the adjustment mechanism 200 also restricts excessive movement, reducing the tearing of skin in the operation. In addition, most parts of the adjustment mechanism 200 are also made of biocompatible material, such that the use of the adjustment mechanism 200 in the surgery does not cause any undesirable effects to the patient. For example, the materials that are suitable include titanium and polyether ether ketone (PEEK). It will be appreciated that the structure of the adjustment mechanism 200 may be made of other materials.

In an embodiment, the surgical tool 212 may comprise a tactile sensor (not shown) communicatively coupled to the processor to detect pressure change on the surgical tool 212. This may enhance the accuracy of the processor in detecting the depth of the surgical tool 212 inside the patient's body and in detecting the lesion.

FIG. 2C shows two perspective views illustrating the use of a tool holder 224 of the adjustment mechanism 200 of FIG. 2A. Here, the tool holder 224 is detachable from the platform 206. The structure of the tool holder 224 includes the ball joint compliance 210 and a plurality of supporting structures 226 extending radially outward from the ball joint compliance 210, linking the ball joint compliance 210 with the annular ring of the platform 206. An engagement mechanism, represented as catches 228, is used for detachably fastening the tool holder 224 to the platform 206.

As shown in the first arrangement (the left diagram on FIG. 2C), the tool holder 224 is attached to the platform 206 when the platform 206 is moved to adjust the angular orientation of the surgical tool 212. The tilting of the surgical tool 212 is shown by arrow 218. As shown in the second arrangement (the right diagram on FIG. 2C), if further insertion is required beyond the insertion depth allowed by the stopper 220, the tool holder 224 is detached from the platform 206, e.g. by turning the tool holder 224 in the clockwise or anticlockwise direction, and lowered onto the patient's body, as shown by arrow 230.

The tool holder 224 can then be mounted on the patient's body, and the plunger is actuated by the actuator to hold and further insert the surgical tool 212 into the patient's body, as shown by arrow 232. The tool holder 224 thus allows the surgical tool 212 to be inserted into the patient's body to a greater depth, providing flexibility in the type of operation that can be performed using the adjustment mechanism 200.

FIG. 2D shows an adjustment of the surgical tool 212 using the adjustment mechanism 200 of FIG. 2A. As shown in FIG. 2D, there are three planes involved in the adjustment of the surgical tool 212 from a first angular orientation 234a to a second angular orientation 234b, i.e. a target plane 236a, a pivot point plane 236b and an adjustment mechanism plane 236c.

The coordinates of the lesion in the 3D voxel grids is denoted by S₀. The pivot point coordinates P₀ for the insertion of the surgical tool 212 has been determined using the system 101 of FIGS. 1A and 1B. During the operation, the tip of the surgical tool 212 is placed on the pivot point.

The trajectory of the surgical tool 212 forming a substantially straight line with the coordinates S₀ and P₀ has also been determined by the system 101 of FIGS. 1A and 1B. In an embodiment, using both coordinates S₀ and P₀, the processor calculates aligning coordinates P₁ on the adjustment mechanism plane 236c. Based on the aligning coordinates P₁, the processor controls the adjustment mechanism 200 to adjust the surgical tool 212 at the adjustment mechanism plane 236c from the first angular orientation 234a to the second angular orientation 234b, such that the longitudinal axis of the surgical tool 212 passes through the aligning coordinates P₁ in the second angular orientation 234b. This can be done, for example, by moving the platform 206 laterally such that the ball joint compliance 210 is at the coordinates P₁. In the second angular orientation 234b, the longitudinal axis of the surgical tool 212 is aligned with the lesion and the pivot point. The steps of determining the coordinates S₀ and P₀, calculating the aligning coordinates P₁ and adjusting the surgical tool 212 to align with the aligning coordinates P₁ may be repeated automatically to correct any errors until the longitudinal axis of the surgical tool 212 substantially aligns with the lesion.

FIG. 2E shows another example configuration of the adjustment mechanism 200 of FIG. 2A during operation. Here, the adjustment mechanism 200 is in an inverted position above the patient's body, with the base 202 held stationary by an articulated arm fixture 238. In this configuration, the platform 206 is elevated with respect to the patient's body and can be adjusted to move laterally as shown by arrow 240a, 240b when the surgical tool 212 is held loosely by the plunger and the ball joint compliance 210, allowing the surgical tool 212 to pivot or swivel freely about an insertion point 242 on a body surface. The tilting of the surgical tool 212 is shown by arrow 244. Upon confirmation of the angular orientation and the depth of insertion, the position of the ball joint compliance 210 is locked and the plunger is actuated to hold and insert the surgical tool 212 into the body.

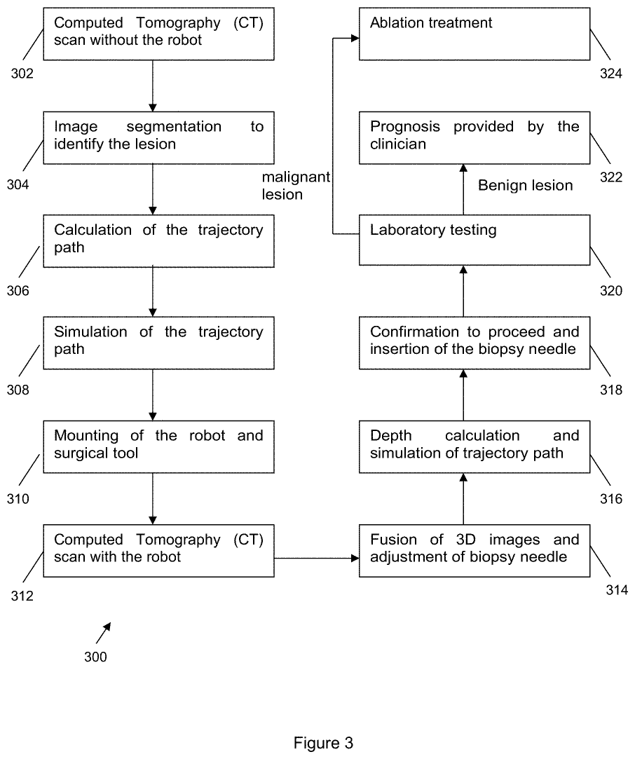

FIG. 3 shows a flowchart 300 illustrating a treatment process of a lesion using the set-up 100 of FIGS. 1A and 1B.

At step 302, the 3D imaging device 106 captures a CT scan image of the body 102 in the absence of the robot 110 to produce the preliminary 3D image and the image data of the preliminary 3D image is transferred to the processor through DICOM.

At step 304, the processor performs image segmentation process on the preliminary 3D image to identify the images of the occlusions and lesion inside the body 102 based on the shades on the preliminary 3D image. Based on the lesion image, the processor extracts the location data of the lesion by calculating centroid coordinates of the lesion in 3D voxel grids.

At step 306, the processor determines at least one trajectory for a biopsy needle to collect samples of the lesion. For example, the AI software identifies all the important organ or arteries on the preliminary 3D image and determines a tool insertion point on the body surface for the insertion of the surgical tool 104. In an embodiment, the tool insertion point has the shortest distance between the body surface and the lesion. Alternatively or in addition, the tool insertion point forms a line with the lesion which bypasses the occlusions inside the body 102.

At step 308, the processor simulates the trajectory of the biopsy needle into the body 102. The clinician can be guided by the simulated trajectory to decide an anatomically ideal point to insert the surgical tool 104 such that the possibility of induced complication due to the insertion of the biopsy needle can be reduced.

At step 310, the robot 110 together with the biopsy needle is mounted on the patient's body 102.

At step 312, the 3D imaging device 106 captures a CT scan image of the body 102 with the robot 110 and the biopsy needle, to produce the real-time 3D image. The image data of the real-time 3D image is transferred to the processor through DICOM.

At step 314, the AI software performs image fusion of the preliminary 3D image and the real-time 3D image for calibration of the robot 110 with the fused 3D images, followed by an adjustment of the angular orientation of the biopsy needle using the robot 110 for alignment with the selected trajectory. During this process, the AI software will take into account the change of the lesion location due to reasons such as a change in the patient positioning, chest movement during breathing, etc. This may advantageously allow the AI software to obtain the precise location of the lesion and to determine an accurate trajectory according to the change of the lesion location.

At step 316, the processor calculates the depth of insertion of the biopsy needle from the skin surface to the lesion. The processor further simulates the trajectory of the biopsy needle into the body 102 according to the calculated depth.

At step 318, the clinician does a final confirmation and proceeds with the insertion of the biopsy needle into the body 102. In some cases, multiple samples from the lesion may be taken according to the coordinates which have been determined by the processor for sample collection to produce a more accurate test result.

At step 320, the samples taken are sent for laboratory testing to determine the malignancy of the lesion.

If the laboratory testing finds that the lesion is a benign lesion, ablation of the lesion may not be required and at step 322, the clinician may provide prognosis of the condition, including advising the patient to repeat the biopsy at a later time for confirmation of the results.

If the laboratory testing finds that the lesion is a malignant lesion, at step 324, the clinician proceeds with providing an ablation treatment of the lesion using the robot 110. The planning for insertion of the ablation needle is performed using the same steps as the biopsy process from steps 302 through 318.

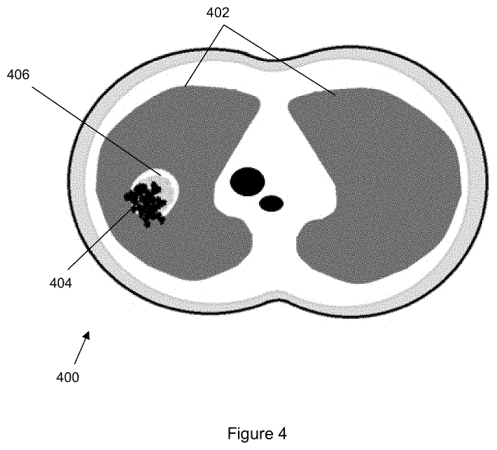

FIG. 4 shows a transverse plane view 400 of lungs 402 in a CT scan. As shown in the CT scan, the left lung 402 has two spots with lesions, represented by a dark-shaded area 404 and a light-shaded area 406 on the CT scan. The AI software can identify the lesions based on the colour difference, as further described below. Biopsy process can be performed to determine the malignancy of these lesions.

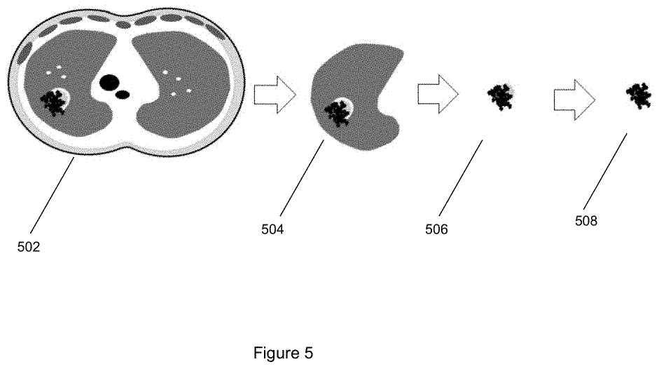

FIG. 5 shows the segmentation process of a CT scan using the system 101 of FIGS. 1A and 1B. The segmentation process is divided into 3 steps with the first image 502 showing the CT scan of the lungs 402 of FIG. 4 having lesions in the left lung 402. The processor receives the CT scan from the 3D imaging device 106 and processes the CT scan to identify the image of the left lung 402 which has a lesion. The image of left lung 402 is segmented from the CT scan to generate the second image 504.

The processor further identifies the lesions according to the difference in the shades and segments the image of the lesions from the second image to produce the third image 506. The processor further identifies the dark-shaded area 404 on the third image 506 and segments the dark-shaded area 404 from the third image to produce the fourth image 508. The segmentation process produces the segmented lesion image represented by the dark-shaded area 404. The location data of the lesion can be accurately extracted by the processor from the lesion image and the lesion is set as the target in a biopsy or ablation treatment based on the extracted location data.

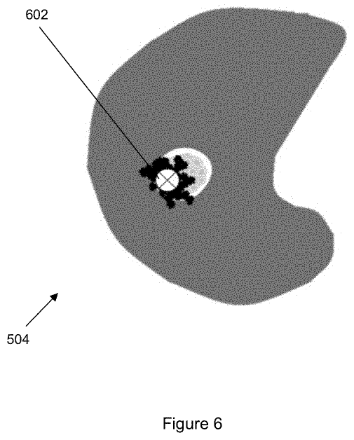

FIG. 6 shows the centroid location 602 of the lesion in a segmented view 504 of FIG. 5. In an embodiment, the centroid location 602 of the lesion in the 3D voxel grids is calculated by the processor using an algorithm applying the First Moment of Volume Integral. The formulae for the calculation of centroid coordinates at each dimension are provided below.

$$\overline{x} = \frac{{\int }_{V} dV * x}{V} \overline{y} = \frac{{\int }_{V} dV * y}{V} \overline{z} = \frac{{\int }_{V} dV * z}{V}$$

In the integral calculation, all the volumes along the x, y and z coordinates are respectively summed up and divided by the total volume of the lesion to obtain the rate of change of the volume from one end to another end along each axis. The rate of change of the volume at any point is equivalent to the cross sectional area perpendicular to the axis. The formula is able to take into account the variation in the cross sectional area in determining the average coordinates of each dimension.

It will be appreciated that the centroid coordinates can also be determined using other mathematical methods such as the method of composite parts.

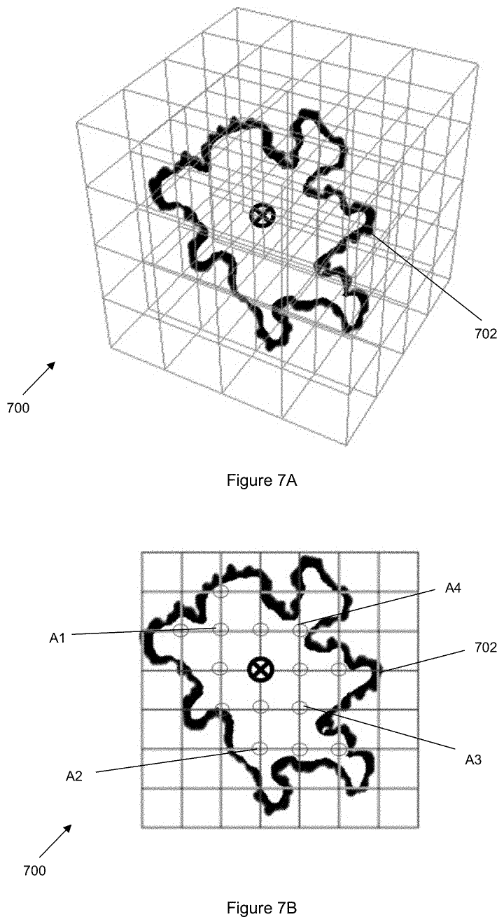

FIGS. 7A and 7B show illustrations 700 of a lesion 702 in 3D voxel grids according to an example embodiment. Using the centroid of the lesion 702, the processor determines the optimum grid size based on the volume of the lesion 702 and proposes locations around the centroid (i.e. Locations A1, A2, A3, A4) to either collect samples of the lesion during a biopsy treatment or to perform ablation. The use of 3D voxel grids allows accurate cell-to-cell distance to be determined and enables the clinician to perform treatments at accurate locations to deliver best results.

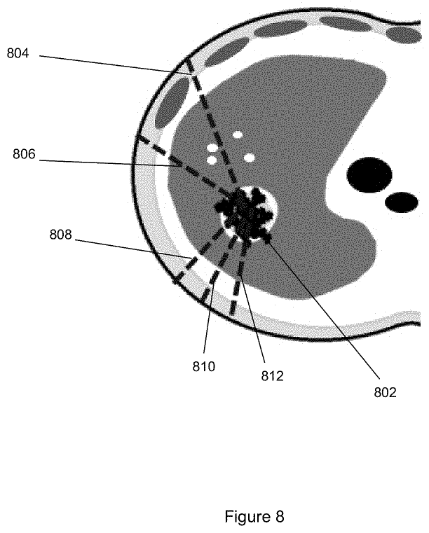

FIG. 8 shows the determination of trajectories of the surgical tool 104 to strike a lesion 802 according to an example embodiment. The processor calculates multiple trajectories to strike the lesion, represented as straight lines 804, 806, 808, 810, 812. The processor then simulates trajectories of the surgical tool 104 toward the lesion 802 in accordance with the determined trajectories. Based on the simulations, the processor applies algorithm to select the optimum trajectory. In this instance, the trajectory 808 is selected as the optimum trajectory. The selected trajectory is used for alignment of the surgical tool 104 in an operation as described above.

Embodiments of the present invention provide a system and method for determining a trajectory of an elongated tool. The surgical process using the system and method can be simplified with minimum human inputs. The locations of the lesions and organs in the body can be determined accurately and the system calculates the optimum trajectory of the surgical tool based on the determined locations. This may advantageously reduce human errors and avoid injuring the organs during the insertion of the surgical tool, thus improving the successful rate of the surgery.

FIG. 9 depicts an exemplary computing device 900, hereinafter interchangeably referred to as a computer system 900, where one or more such computing devices 900 may be used to determine a trajectory of an elongated tool. The exemplary computing device 900 can be used to implement the system 101 shown in FIGS. 1A and 1B. The following description of the computing device 900 is provided by way of example only and is not intended to be limiting.

As shown in FIG. 9, the example computing device 900 includes a processor 907 for executing software routines. Although a single processor is shown for the sake of clarity, the computing device 900 may also include a multi-processor system. The processor 907 is connected to a communication infrastructure 906 for communication with other components of the computing device 900. The communication infrastructure 906 may include, for example, a communications bus, cross-bar, or network.

The computing device 900 further includes a main memory 908, such as a random access memory (RAM), and a secondary memory 910. The secondary memory 910 may include, for example, a storage drive 912, which may be a hard disk drive, a solid state drive or a hybrid drive, and/or a removable storage drive 917, which may include a magnetic tape drive, an optical disk drive, a solid state storage drive (such as a USB flash drive, a flash memory device, a solid state drive or a memory card), or the like. The removable storage drive 917 reads from and/or writes to a removable storage medium 977 in a well-known manner. The removable storage medium 977 may include magnetic tape, optical disk, non-volatile memory storage medium, or the like, which is read by and written to by removable storage drive 917. As will be appreciated by persons skilled in the relevant art(s), the removable storage medium 977 includes a computer readable storage medium having stored therein computer executable program code instructions and/or data.

In an alternative implementation, the secondary memory 910 may additionally or alternatively include other similar means for allowing computer programs or other instructions to be loaded into the computing device 900. Such means can include, for example, a removable storage unit 922 and an interface 950. Examples of a removable storage unit 922 and interface 950 include a program cartridge and cartridge interface (such as that found in video game console devices), a removable memory chip (such as an EPROM or PROM) and associated socket, a removable solid state storage drive (such as a USB flash drive, a flash memory device, a solid state drive or a memory card), and other removable storage units 922 and interfaces 950 which allow software and data to be transferred from the removable storage unit 922 to the computer system 900.

The computing device 900 also includes at least one communication interface 927. The communication interface 927 allows software and data to be transferred between computing device 900 and external devices via a communication path 926. In various embodiments of the inventions, the communication interface 927 permits data to be transferred between the computing device 900 and a data communication network, such as a public data or private data communication network. The communication interface 927 may be used to exchange data between different computing devices 900 which such computing devices 900 form part an interconnected computer network. Examples of a communication interface 927 can include a modem, a network interface (such as an Ethernet card), a communication port (such as a serial, parallel, printer, GPIB, IEEE 1394, RJ45, USB), an antenna with associated circuitry and the like. The communication interface 927 may be wired or may be wireless. Software and data transferred via the communication interface 927 are in the form of signals which can be electronic, electromagnetic, optical or other signals capable of being received by communication interface 927. These signals are provided to the communication interface via the communication path 926.

As shown in FIG. 9, the computing device 900 further includes a display interface 902 which performs operations for rendering images to an associated display 950 and an audio interface 952 for performing operations for playing audio content via associated speaker(s) 957.

As used herein, the term “computer program product” may refer, in part, to removable storage medium 977, removable storage unit 922, a hard disk installed in storage drive 912, or a carrier wave carrying software over communication path 926 (wireless link or cable) to communication interface 927. Computer readable storage media refers to any non-transitory, non-volatile tangible storage medium that provides recorded instructions and/or data to the computing device 900 for execution and/or processing. Examples of such storage media include magnetic tape, CD-ROM, DVD, Blu-ray™ Disc, a hard disk drive, a ROM or integrated circuit, a solid state storage drive (such as a USB flash drive, a flash memory device, a solid state drive or a memory card), a hybrid drive, a magneto-optical disk, or a computer readable card such as a PCMCIA card and the like, whether or not such devices are internal or external of the computing device 900. Examples of transitory or non-tangible computer readable transmission media that may also participate in the provision of software, application programs, instructions and/or data to the computing device 900 include radio or infra-red transmission channels as well as a network connection to another computer or networked device, and the Internet or Intranets including e-mail transmissions and information recorded on Websites and the like.

The computer programs (also called computer program code) are stored in main memory 908 and/or secondary memory 910. Computer programs can also be received via the communication interface 927. Such computer programs, when executed, enable the computing device 900 to perform one or more features of embodiments discussed herein. In various embodiments, the computer programs, when executed, enable the processor 907 to perform features of the above-described embodiments. Accordingly, such computer programs represent controllers of the computer system 900.

Software may be stored in a computer program product and loaded into the computing device 900 using the removable storage drive 917, the storage drive 912, or the interface 950. The computer program product may be a non-transitory computer readable medium. Alternatively, the computer program product may be downloaded to the computer system 900 over the communications path 926. The software, when executed by the processor 907, causes the computing device 900 to perform functions of embodiments described herein.

It is to be understood that the embodiment of FIG. 9 is presented merely by way of example. Therefore, in some embodiments one or more features of the computing device 900 may be omitted. Also, in some embodiments, one or more features of the computing device 900 may be combined together. Additionally, in some embodiments, one or more features of the computing device 900 may be split into one or more component parts.

When the computing device 900 is configured to determine a trajectory of an elongated tool, the computing system 900 will have a non-transitory computer readable medium having stored thereon an application which when executed causes the computing system 900 to perform steps comprising: receiving a preliminary 3-dimensional (3D) image of a body, the body comprising an opaque body surface, a target and at least one occlusion between the body surface and the target; processing the preliminary 3D image of the body from the 3D imaging device to obtain location data of the target, body surface and at least one occlusion; and based on a location of the target relative to the body surface and at least one occlusion, determining at least one trajectory for the elongated tool to strike the target.

It will be appreciated by a person skilled in the art that numerous variations and/or modifications may be made to the present invention as shown in the specific embodiments without departing from the spirit or scope of the invention as broadly described. The present embodiments are, therefore, to be considered in all respects to be illustrative and not restrictive.

1. A system for determining a trajectory of an elongated tool, the system comprising:
a memory module configured to receive imaging data of a preliminary 3-dimensional (3D) image of a body from a 3D imaging device, the body comprising an opaque body surface, a target and at least one occlusion between the body surface and the target;
a processor communicatively coupled with the memory module, wherein the processor is configured to:
process the preliminary 3D image of the body from the 3D imaging device to obtain location data of the target, body surface and at least one occlusion; and
based on a location of the target relative to the body surface and at least one occlusion, determine at least one trajectory for the elongated tool to strike the target.

2. The system as claimed in claim 1, wherein the processor is further configured to determine at least one tool insertion point on the body surface to determine the at least one trajectory for the elongated tool to strike the target.

3. The system as claimed in claim 2, wherein the processor is further configured to determine the insertion point on the body surface having the shortest distance between the body surface and the target.

4. The system as claimed in claim 2, wherein the processor is further configured to determine the insertion point on the body surface such that a line between the insertion point and the target bypasses the at least one occlusion.

5. The system as claimed in claim 1, wherein the processor is further configured to determine coordinates of a centroid of the target to obtain location data of the target.

6. The system as claimed in claim 1, further comprising a display device coupled to the processor, wherein the processor is further configured to simulate a trajectory of the elongated tool based on the determined at least one trajectory for display on the display device.

7. A system for striking a target using an elongated tool, the system comprising:
a 3D imaging device;
the system as claimed in claim 2 connected to the 3D imaging device;
an adjustment mechanism configured to adjust an angular orientation of the elongated tool relative to the insertion point; and
an actuator coupled to the adjustment mechanism for moving the adjustment mechanism according to signals received from the processor,
wherein the 3D imaging device is further configured to capture a real-time 3D image of the body and the elongated tool,
wherein the processor is further configured to control the adjustment mechanism to align a longitudinal axis of the elongated tool with a selected trajectory based on the real-time 3D image, the processor further configured to calculate a striking distance between the insertion point and the target based on location data of the insertion point and the target; and
wherein the actuator is configured to drive the elongated tool toward the target based on the angular orientation of the elongated tool at alignment and the calculated striking distance.

8. The system as claimed in claim 7, wherein the processor is further configured to associate the real-time 3D image with the preliminary 3D image, to align the elongated tool to the selected trajectory.

9. The system as claimed in claim 7, wherein the 3D imaging device comprises at least one selected from a group consisting of a magnetic resonance imaging (MRI) machine, a computerized tomography (CT) scanner and a fluoroscope.

10. The system as claimed in claim 7, wherein the adjustment mechanism comprises a base and a platform, wherein the platform is configured to be parallel to the base.

11. The system as claimed in claim 10, wherein the adjustment mechanism further comprises a plurality of arms linking the base with the platform, the plurality of arms being configured to move the platform along a plane parallel to the base to adjust the angular orientation of the elongated tool relative to the insertion point.

12. The system as claimed in claim 10, wherein the platform comprises a ball joint compliance for supporting the elongated tool, the ball joint compliance comprising a hole configured to allow sliding movement of the elongated tool therethrough.

13. The system as claimed in claim 10, wherein the adjustment mechanism further comprises a tool holder detachable from the platform.

14. A method for determining a trajectory of an elongated tool, the method comprising the steps of:
receiving a preliminary 3-dimensional (3D) image of a body, the body comprising an opaque body surface, a target and at least one occlusion between the body surface and the target;
processing the preliminary 3D image of the body from the 3D imaging device to obtain location data of the target, body surface and at least one occlusion; and
based on a location of the target relative to the body surface and at least one occlusion, determining at least one trajectory for the elongated tool to strike the target.

15. The method as claimed in claim 14, wherein determining the at least one trajectory for the elongated tool to strike the target comprises determining at least one tool insertion point on the body surface.

16. The method as claimed in claim 15, wherein determining the at least one tool insertion point on the body surface comprises determining the insertion point having the shortest distance between the body surface and the target.

17. The method as claimed in claim 15, wherein determining the at least one tool insertion point on the body surface comprises determining the insertion point on the body surface such that a line between the insertion point and the target bypasses the at least one occlusion.

18. The method as claimed in claim 14, wherein processing the preliminary 3D image of the body to obtain location data of the target comprises determining coordinates of a centroid of the target.

19. The method as claimed in claim 14, further comprising the step of simulating a trajectory of the elongated tool based on the determined at least one trajectory for display on a display device.

20. A method of striking a target using an elongated tool, the method comprising the steps of:
determining at least one trajectory for the elongated tool to strike the target using the method as claimed in claim 15;
receiving a real-time 3D image of the body and the elongated tool;
aligning a longitudinal axis of the elongated tool with a selected trajectory based on the real-time 3D image;
calculating a striking distance between the insertion point and the target based on location data of the insertion point and the target; and
advancing the elongated tool toward the target according to the calculated distance.

21. (canceled)
